(19) United States
(12) Patent Application Publication
(10) Pub. No.: US 2023/0381501 A1
(43) Pub. Date: Nov. 30, 2023

(54) LEADS FOR CARDIAC CONDUCTION SYSTEM WITH DEFIBRILLATION CAPABILITY

(71) Applicant: Singular Medical (USA) Inc., Irvine, CA (US)

(72) Inventors: Martin Tze, Irvine, CA (US); Scott Hayden, Irvine, CA (US); Yongxing Zhang, Irvine, CA (US); Entao Liu, Irvine, CA (US); Ryan Bauer, Irvine, CA (US); Lichuan Ping, Irvine, CA (US)

(21) Appl. No.: 17/811,524

(22) Filed: Jul. 8, 2022

Related U.S. Application Data
(63) Continuation-in-part of application No. 17/804,705, filed on May 31, 2022.

(51) Int. Cl. A61N 1/05

(52) U.S. Cl. CPC A61N 1/0563; A61N 1/0573

(57) ABSTRACT

A lead for a cardiac conduction system. The lead includes a lead body; a distal end including a first electrode configured to be inserted into a portion of a ventricular septum; a second electrode coupled to the lead body; a fixation element configured to fix the lead to the portion of the ventricular septum; and a proximal end. The lead further includes a shocking coil coupled to the lead body and spaced away from the second electrode.

### CROSS-REFERENCE

This disclosure is a continuation-in-part application that claims the benefit of U.S. application Ser. No. 17/804,705, filed May 31, 2022, which is incorporated herein by reference.

### FIELD

This disclosure relates generally to systems, methods, and designs of lead(s) for cardiac conduction system pacing. More specifically, the disclosure relates to systems and designs of lead(s) for cardiac conduction system pacing that have defibrillation capability, and relates to methods of implanting lead(s) in a ventricular septum and right ventricle using a delivery system including a catheter.

### BACKGROUND

An implantable pulse generator (IPG) (e.g., an implantable pacemaker, an implantable cardioverter-defibrillator, etc.) is a medical device powered by a battery, contains electronic circuitry having a controller, and delivers and regulates electrical impulses to an organ or a system such as the heart, the nervous system, or the like. A lead is a thin, flexible, electrical wire connecting a device such as the IPG to a target such as the organ or system, transmits electrical impulses (e.g., a burst of energy) from the device to the target, and/or senses or measures the potential or the voltage from the target. A catheter is a tubular medical device for insertion into canals, vessels, passageways, or body cavities usually to keep a passage open to facilitate the delivery of e.g., a lead or leads during a surgical procedure. The process of inserting a catheter is “catheterization”. The conduction system of the heart consists of cardiac muscle cells and conducting fibers that are specialized for initiating impulses and conducting the impulses through the heart. The cardiac conduction system initiates the normal cardiac cycle, coordinates the contractions of cardiac chambers, and provides the heart its automatic rhythmic beat. Conduction system pacing (CSP) is a technique of pacing that involves implantation of pacing leads along different sites or pathways of the cardiac conduction system and includes His-bundle pacing, left bundle branch pacing, right bundle branch pacing, and/or bilateral pacing (pacing both the left bundle branch and the right bundle branch).

### SUMMARY

This disclosure relates generally to systems, methods, and designs of lead(s) for cardiac conduction system pacing. More specifically, the disclosure relates to systems and designs of lead(s) for cardiac conduction system pacing that have defibrillation capability, and relates to methods of implanting lead(s) in a ventricular septum and right ventricle using a delivery system including a catheter.

In an embodiment, a lead for cardiac conduction system pacing that has defibrillation capability is disclosed. The lead includes a lead body. The lead also includes a distal end including a first electrode configured to be inserted into a portion of a ventricular septum, and a shocking coil mounted on the lead body and spaced away from the second electrode positioned in the right ventricle. The lead further includes a proximal end. In an embodiment, the lead can further include a second electrode and a fixation element configured to fix the lead to the portion of the ventricular septum.

In an embodiment, a method of implanting a lead using a delivery system is disclosed. The method includes inserting a catheter to reach a septum, positioning the catheter against the septum, inserting the lead through an orifice of the catheter extending from a distal end of the catheter to a proximal end of the catheter, rotating a lead body of the lead to engage a helix electrode of the lead to the septum, and removing the catheter.

Embodiments disclosed herein can provide a lead(s) that can be “deep” seated into the ventricular septum (e.g., inserted inside the ventricular septum in an adequate distance, e.g., the lead body being partially inside the tissue of the ventricular septum) to electrically capture the cardiac conduction system (e.g., to reach the pathway such as the LBB from the cavity of the right ventricle). Embodiments disclosed herein can also provide a catheter and lead(s) that can be more atraumatic and easier to be delivered to a desired location. Embodiments disclosed herein can further provide a catheter and lead(s) that can minimize the trauma to the heart tissue and have stable electrical performance.

Other features and aspects will become apparent by consideration of the following detailed description and accompanying drawings.

### BRIEF DESCRIPTION OF THE DRAWINGS

References are made to the accompanying drawings that form a part of this disclosure and which illustrate the embodiments in which systems and methods described in this specification can be practiced.

FIGS. 1A-1E are side views of a lead, according to an embodiment.

FIG. 2 is a side view of a lead, according to another embodiment.

FIG. 3 is a side view of a lead, according to another embodiment.

FIG. 4 illustrates a lead for fixation, according to an embodiment.

FIG. 5 illustrates a lead implanted in the ventricular septum and right ventricle, according to an embodiment.

Particular embodiments of the present disclosure are described herein with reference to the accompanying drawings; however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. In this description, as well as in the drawings, like-referenced numbers represent like elements that may perform the same, similar, or equivalent functions.

### DETAILED DESCRIPTION

This disclosure relates generally to systems, methods, and designs of lead(s) for cardiac conduction system pacing. More specifically, the disclosure relates to systems and designs of lead(s) for cardiac conduction system pacing that have defibrillation capability, and relates to methods of implanting lead(s) into a ventricular septum and the right ventricle using a delivery system including a catheter.

As defined herein, the phrase “distal” may refer to being situated away from a point of attachment (e.g., to a device such as the implantable pulse generator) or from an operator (e.g., a physician, a user, etc.). A distal end of a lead or a catheter may refer to an end of the lead or the catheter that is away from the operator or from a point of attachment to the IPG.

As defined herein, the phrase “proximal” may refer to being situated nearer to a point of attachment (e.g., to a device such as the implantable pulse generator) or to an operator (e.g., a physician, a user, etc.). A proximal end of a lead or a catheter may refer to an end of the lead or the catheter that is close to the operator or to a point of attachment to the IPG.

As defined herein, the phrase “French” may refer to a unit to measure the size (e.g., diameter or the like) of device such as a catheter, a lead, etc. For example, a round catheter or lead of one (1) French has an external diameter of ⅓ millimeters. For example, if the French size is 9, the diameter is 9/3=3.0 millimeters.

As defined herein, the phrase “helix” may refer to (e.g., an object) having a three-dimensional shape like that of a wire wound (e.g., in a single layer) around a cylinder or cone, as in a corkscrew or spiral staircase. The phrase “linear” may refer to being arranged in or extending straightly or nearly straightly.

As defined herein, the phrase “conductive” may refer to electrically conductive.

As defined herein, the phrase “septum” may refer to a partition separating two chambers, such as that between the chambers of the heart. Septum can be atrial septum and/or ventricular septum. The phrase “ventricular septum” or “interventricular septum” (“IVS”) may refer to a partition separating two ventricular chambers. The phrase “right ventricular septum” may refer to the ventricular septum where the RBB is located, while “left ventricular septum” may refer to the ventricular septum where the LBB is located.

As defined herein, the phrase “pacing” may refer to depolarization of the atria or ventricles, resulting from an impulse delivered (e.g., at desired voltage(s) for a desired duration, or the like) from a device (such as a pulse generator) down a lead to the heart via myocardium or directly via the cardiac conduction system. The phrase “sensing” may refer to detection by the device of intrinsic atrial or ventricular or conduction system electrical signals that are conducted up a lead. It will be appreciated that each of the electrodes described herein can be configured as a pacing electrode and/or a sensing electrode and/or a combination thereof. It will also be appreciated that each of the electrodes described herein can be configured as anode and/or cathode and/or a combination thereof.

As defined herein, the phrase “conduction system pacing” or “CSP” may refer to a therapy that involves the placement of pacing leads along different sites or pathways to electrically capture the cardiac conduction system with the intent of overcoming sites of atrioventricular conduction disease and delay, thereby providing a pacing solution that results in more synchronized biventricular activation. Lead placement for CSP can be targeted at the bundle of His, known as His-bundle pacing (HBP), at the region of the left bundle branch (LBB), known as LBB pacing (LBBP), or at the region of the right bundle branch (RBB), known as RBB pacing (RBBP) or both at the regions of RBB and LBB for Bi-lateral Bundle Branch Pacing (BBBP). Compared with conventional right ventricular (RV) pacing or biventricular (RV and left ventricular (LV)) pacing, where RV apical pacing lead and/or LV epicardial lead are implanted, the lead for CSP is placed through the septum e.g., closer to the His-bundle, the LBB, and/or the RBB. As such, the design, function, and purpose of the lead(s) for cardiac conduction system pacing are different from those of the lead(s) for RV and/or LV pacing. It will be appreciated that ventricular pacing (e.g., RV pacing or the like) may be un-physiological and may result in adverse outcomes of mitral and/or tricuspid regurgitations, atrial fibrillation, heart failure, and/or pacing induced cardiomyopathy. CSP can be physiological pacing that can results in electrical-mechanical synchronization to mitigate chronic clinical detrimental consequence including e.g., pacing induced cardiomyopathy. It will also be appreciated that CSP indications may include e.g., a high burden of ventricular pacing being necessary (e.g., permanent atrial fibrillation with atrioventricular block, slowly conducted atrial fibrillation, pacing induced cardiomyopathy, atrioventricular node ablation, etc.); sick sinus syndrome, when atrioventricular node conduction diseases exist; and/or an alternative to biventricular pacing in heart failure patients with bundle branch block, narrow QRS and PR prolongation, biventricular pacing no-responders or patients need biventricular pacing cardiac resynchronization therapy upgrade, or the like.

Some embodiments of the present application are described in detail with reference to the accompanying drawings so that the advantages and features of the present application can be more readily understood by those skilled in the art. The terms “near”, “far”, “top”, “bottom”, “left”, “right”, and the like described in the present application are defined according to the typical observation angle of a person skilled in the art and for the convenience of the description. These terms are not limited to specific directions.

Processes described herein may include one or more operations, actions, or functions depicted by one or more blocks. It will also be appreciated that although illustrated as discrete blocks, the operations, actions, or functions described as being in various blocks may be divided into additional blocks, combined into fewer blocks, or eliminated, depending on the desired implementation. Any features described in one embodiment may be combined with or incorporated/used into the other embodiment, and vice versa. The scope of the disclosure should be determined by the appended claims and their legal equivalents, rather than by the examples given herein. For example, the steps recited in any method claims may be executed in any order and are not limited to the order presented in the claims. Moreover, no element is essential to the practice of the disclosure unless specifically described herein as “critical” or “essential.”

As discussed above, conduction system pacing (CSP) is a technique of pacing that involves implantation of pacing leads along different site(s) or pathway(s) to electrically capture the cardiac conduction system and includes, for example, His-bundle pacing, left bundle branch pacing, right bundle branch pacing, and/or bilateral pacing (pacing both the left bundle branch and the right bundle branch). CSP can be physiological pacing that can results in electrical-mechanical synchronization to mitigate chronic clinical detrimental consequence including e.g., pacing induced cardiomyopathy. Prior CPS leads, however, do not have defibrillating capability to treat certain conditions, for example, tachyarrhythmia. While there are devices with defibrillating capability, such as, cardiac resynchronization therapy defibrillator (CRT-D) devices, such devices have complex designs that require positioning of the leads at specific positions to reach either the anterior interventricular vein or other branch to pace the left ventricle. Not only is the positioning of the lead in the existing CRT-D devices complicated, due to the anatomy of the patient, such devices may not provide the proper therapy for the patient, e.g., 25% of patients may not respond to the CRT-D device for therapy.

As such, in an embodiment as disclosed herein, a lead for cardiac conduction system pacing is provided that includes a shocking coil such that the lead has defibrillation capabilities and/or adjustability of pacing vectors, e.g., allows different configurations for pacing, sensing, shocking, etc. Thus, such an improved lead may have benefits for heart failure patients that use CRT-D devices and/or cardiac resynchronization therapy devices in general that is easier to use and provides additional benefits for those who need cardiac resynchronization therapy.

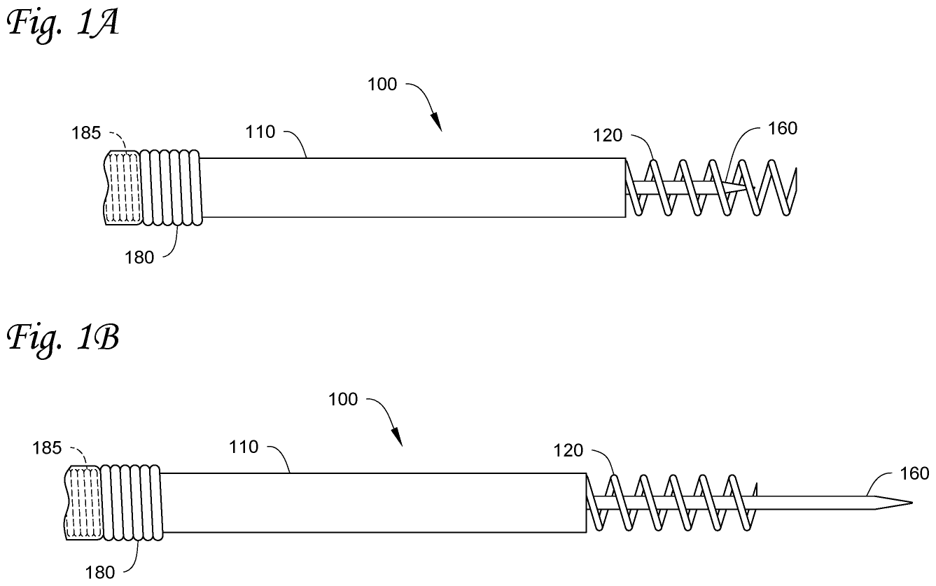

FIGS. 1A-1E illustrate a lead having a shocking coil, according to an embodiment. It is appreciated that while the lead of FIGS. 1A-1E illustrates the first electrode as a linear electrode and the second electrode as a helix electrode, the description of the lead is not intended to limit the scope of the disclosure but provided as an illustrative example. Rather, other configurations of the lead can be used, for example, as disclosed in U.S. application Ser. No. 17/804,705 to Singular Medical Inc., filed May 31, 2022, which is incorporated herein by reference. For example, in an embodiment, the lead also includes a distal end having an electrode (e.g., a helix electrode or the like). The lead body includes a non-conductive spacer proximal to the helix electrode, an outer electrode (e.g., a ring electrode disposed around the lead body made of titanium, platinum, platinum-iridium alloy, or the like, and coated for increased surface area and/or having a fractal coating, e.g., TiN or IrOx) proximal to the spacer, an electrode coil (e.g., the coil of the ring electrode which can be made of MP35N and/or silver) extends to the proximal end of the lead and connects to a connector (not shown) at the proximal end of the lead body, and an inner electrode (e.g., a coupler or the like) that connects the electrode coil to the outer electrode.

As shown in FIGS. 1A-1E, the lead 100 for cardiac conduction system pacing that has defibrillation capability includes a lead body 110 and a distal end including a first electrode 160 configured to be inserted into a portion of the ventricular septum. The lead 100 also includes a shocking coil 180 mounted on the lead body and spaced away from the distal end. The lead 100 further includes a proximal end, which is not shown in the figures. At the proximal end of the lead 100, an implantable pulse generator (IPG) can be provided to generate the bursts of energy for treating the heart, e.g., pacing and/or defibrillation. In an embodiment, as illustrated in FIGS. 1A-1E, the lead 100 further includes a second electrode 120 coupled to the lead body 110, as further discussed below. It is appreciated that the terms coupled, mounted on, etc. are not intended to limit the scope of the disclosure, but are broadly directed to different mechanisms for connecting, directly and/or indirectly, the different components of the lead. For example, the components can be connected mechanically, chemically, e.g., biocompatible adhesive, welded, ultrasonically welded, etc.

The lead body 110 includes an outer layer 111 and an outer coil 122 having windings spaced close to each other. The outer layer 111 can be a multilayered structure that includes one or more layers, including an electrical insulator or insulation layer, a braided layer or gripping layer for rotating the lead 100, a conducting layer, for example, the conduction layer connected to electrical coils or cables, or the like. The outer layer 111 can be made of silicon, polyurethane, ethylene tetrafluoroethylene, polytetrafluoroethylene, and/or other suitable biocompatible material. The windings of the outer coil 122 are connected to or adjacent an inner surface of the outer layer 111. The lead body 110 has a distal end 118. The outer coil 122 extends from a proximal end of the lead body 110 to a location close or proximate to a distal tip of the distal end 118 of lead body 110.

Inside the outer layer 111 near the distal tip of the distal end 118, the second electrode 120 has windings 121 to keep the helix electrode in place. The windings of the second electrode 120 extend outside of the lead body 110, and the portion of the windings of the second electrode 120 that is outside of the lead body 110 may have variable or the same pitches between coils. The second electrode 120 includes a first portion extending distally from the lead body, and a second portion extending distally from the first portion. In an embodiment, a diameter of the windings of the second electrode 120 is larger than a diameter of the windings of the outer coil 122. In an embodiment, the second electrode 120 can have a length that ranges from at or about 2.2 millimeters to at or about 2.6 millimeters, preferably at or about 2.4 millimeters, which is greater than a length of a conventional helix electrode of at or about 1.8 millimeters. In another embodiment, the second electrode 120 can have a length of at or about 4 millimeters. In an embodiment, a proximal portion of the second electrode 120 can be coated with non-conductive material. In another embodiment, the proximal portion of the second electrode 120 is not coated with non-conductive material. In an embodiment, the outer coil 122 is electrically connected to the second electrode 120 so that electrical impulses (e.g., a burst of energy) can be delivered via the outer coil 122 to the second electrode 120 and/or sensing signals can be detected at the second electrode 120 and carried over to a device through the outer coil 122 to/from the IPG (not shown).

In an embodiment, the lead 100 can include a fixation element configured to fix the lead to the portion of the ventricular septum. In the embodiment as illustrated, it is appreciated that the second electrode 120 includes the fixation element to fix the lead to the ventricular septum. For example, since the second electrode 120 is coupled to the lead body (e.g., via windings 121) and includes a helix winding, rotating the lead 100 or the lead body 110 can extend distally or retract proximally the entire lead 100 to or from the portion of the ventricular septum, e.g., the second electrode 120 is configured to engage with the heart tissue or retract from the heart tissue by a relative rotation thereof. In other embodiments, the fixation element can be a separate component of the lead, for example, fixation wings, clips, hooks, helical screws, or the like.

The first electrode 160 extends from the lead body 110 and can include a tapered tip, a rod integral to the tapered tip, and a spacer 150 connected to the first electrode 160. As such, the first electrode 160 can be a linear electrode that is configured to be inserted into a portion of the ventricular septum to electrically capture the cardiac conduction system. In other embodiments, the first electrode 160 can be a helical electrode or the like.

A middle layer 119 extends from a proximal end of the lead body 110 to a portion of the distal end 118 of the lead body 110. The middle layer 119 is connected to or adjacent an inner surface of the outer coil 122. A housing 113 is disposed inside the outer coil 122 and the windings 121 of the second electrode 120 inside the lead body 110. A proximal portion of the housing 113 is disposed between the middle layer 119 and an inner coil 115. A middle portion of the housing 113 is disposed between the outer coil 122 and the windings 121 and the inner coil 115. A distal portion of the housing 113 is disposed between the windings 121 and the spacer 150 or the rod of the first electrode 160. In an embodiment, the housing 113 is made of plastic or biocompatible material.

The inner coil 115 extends from a proximal end of the lead body 110 to a location close to the distal tip of the distal end 118 of lead body 110. In an embodiment, the inner coil 115 is electrically connected to the first electrode 160 so that electrical impulses (e.g., a burst of energy) can be delivered via the inner coil 115 to the first electrode 160 and/or sensing signals can be detected at the first electrode 160 and carried over to a device through the inner coil 115 to/from the IPG (not shown). The inner coil 115 is connected to or adjacent an inner surface of the middle layer 119 and an inner surface of a portion of the housing 113. At the proximal portion of the housing 113 and between windings of inner coil 115, a plurality of drive components (e.g., drive “tooth”) is disposed at, fixed to, and connected to an inner surface of the proximal portion of the housing 113. The drive components are configured to cause the inner coil 115 to extend distally or retract proximally the spacer 150 and the first electrode 160 that connects to the spacer 150. In an embodiment, a terminal pin (not shown) or any suitable control mechanism at the proximal end of the lead 100 can be rotated to drive the inner coil 115 to extend distally or retract proximally the spacer 150 and the first electrode 160 that connects to the spacer 150.

In an embodiment, the spacer 150 can be integral to the first electrode 160 as an electrode (e.g., single polar or bipolar, made of metal or the like). In such embodiment, the spacer 150 portion of the electrode can be coated (to be non-conductive, e.g., with an electrical insulation layer), and the first electrode 160 portion or a distal tip portion of the electrode is not coated for stimulation delivery pacing (e.g., delivering electrical impulses) and/or sensing.

The spacer 150 and the first electrode 160 extends through a cavity inside the housing 113, through a space inside the outer coil 122, and through a space inside the second electrode 120. The spacer 150 has a length that ranges from at or about 4 mm to at or about 12 mm. In an embodiment, the spacer 150 can be made with different length (at or about 4 mm, at or about 6 mm, at or about 8 mm, at or about 12 mm, or the like, to be suitable to electrically capture both the LBB and the RBB with the helix electrode) to correspond to various thicknesses of the ventricular septum. In such embodiment, in operation, ultrasound or the like can be performed to measure the thickness of the ventricular septum and to determine and/or select the desired length of the spacer. It will be appreciated that the phrase “retracted state” of the lead may refer to a state of the lead where the spacer 150 and the first electrode 160 are fully or partially retracted proximally (e.g., a distal end of the first electrode 160 is proximal to a distal end of the second electrode 120). The phrase “extended state” of the lead may refer to a state of the lead where the spacer 150 and the first electrode 160 are fully or partially extended distally (e.g., a distal end of the first electrode 160 is distal to a distal end of the second electrode 120).

The shocking coil 180 is mounted on the lead body 110 by being embedded in or connected to an outer surface of the lead body 110, e.g., mechanically. A supply coil 185 extends from the proximal end of the lead body 100 to a location close to or adjacent the shocking coil 180. In an embodiment, the supply coil 185 is electrically connected to the shocking coil 180 so that electrical impulses (e.g., a burst of energy) can be delivered via the supply coil 185 to the shocking coil 180 and/or sensing signals can be detected at the shocking coil 180 and carried over to a device through the supply coil 185 to/from the IPG (not shown). In an embodiment, the supply coil 185 can be provided between an outer surface of the outer coil 122 and the outer layer 111. An electrically insulating layer can be provided between the supply coil 185 and the outer coil 122. In another embodiment, the supply coil 185 can be provided at any suitable location in the lead body 110. The shocking coil 180 can be made of a biocompatible alloy (for example, Tantalum, titanium, platinum, and/or Pt/Ir or an alloy thereof, or the like) that allows electrical conduction and high voltage shocking, e.g., for defibrillation. The shocking coil 180 can have a wire diameter between about 0.1 mm and about 0.3 mm and a length of about 40 mm to about 100 mm. While not intending to be limiting in scope, but in order to provide an exemplary example, the shocking coil 180 can have a wire diameter of about 0.2 mm and a length of about 57 mm.

The shocking coil 180 can have an outer diameter that is the same, or larger, or smaller than the outer diameter of another portion of the outer surface of the lead body 110, e.g., the remaining portion of the lead body 110. In an embodiment, an outer diameter of the lead body 110 can have a diameter between at or about 2.0 French and at or about 9.0 French, and preferably between at or about 5 to at or about 9 French and the shocking coil 180 can have a diameter preferably between at or about 5 French and at or about 9 French. It is appreciated that since the amount of energy releasable by the shocking coil 180 can be dependent on the available surface area of the shocking coil, as the diameter of the shocking coil is decreased, the length of the shocking coil can be increased. As such, in an embodiment, the shocking coil 180 has a surface area between at or about 350 mm² to at or about 650 mm² and preferably around at or about 500 mm², depending on the length and diameter of the shocking coil, e.g., smaller diameter coils may need to be longer to have the same surface area.

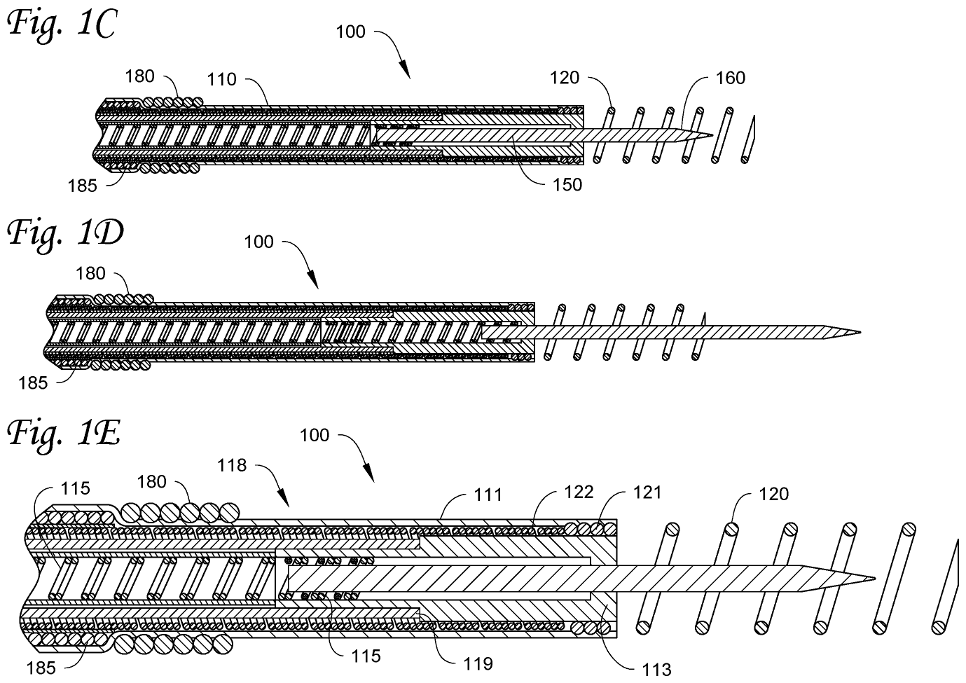

It is appreciated that the shocking coil 180 can be provided on the lead 100 having different configurations and electrical connections to the IPG than discussed above. For example, FIG. 2 illustrates another embodiment of a lead 200 having a single electrode 220 at the distal end. The lead 200 can include the same or similar features as the lead 100 of FIG. 1, and are not discussed in detail with respect to FIG. 2 with respect to the similar features. The lead 200 of FIG. 2 includes the shocking coil 280 provided or disposed around the lead body 210 and the supply coil (or coil conductor) 285 electrically connected to the shocking coil 280. The supply coil 285 can be added as a separate circuit, e.g., using a coil conductor provided around the lead body, so that the shocking coil 280 and supply coil 285 can be added to existing CSP lead designs. In other embodiments, the supply coil 285 and the shocking coil 280 are integrally formed with the lead body 210. In an embodiment, an outer insulation layer 286 is provided around the supply coil 285. The outer insulation layer 286 can be a multilayered structure that includes one or more layers, including an electrical insulator or insulation layer, a braided layer or gripping layer for rotating the lead, a conducting layer, for example, the conduction layer connected to electrical coils or cables, or the like. The outer insulation layer 286 can be made of silicon, polyurethane, or other suitable biocompatible material.

The electrode 220, e.g., a helix electrode, is electrically connected to the IPG by an inner conductor 222 which can be provided in the lead body 210. The inner conductor 222 and/or the supply coil 285 can be made from MP35N and/or silver (multi-filar) coil or cable and can be coated with an ETFE coating or other suitable electrically conductive material. The inner conductor 222 can be separated from the supply coil 285 by an inner insulation layer 211. It is appreciated that the inner insulation layer 211 can be the outer layer (e.g., outer layer 111) in prior designs of the CSP lead, as discussed above. The inner insulation layer 211 can be made of silicon, polyurethane, or other suitable biocompatible material. The inner conductor 222 and the inner insulation layer 211 are provided or disposed internal to the inner diameter of the supply coil 285 and the shocking coil 280. As such, the lead 200 can be configured to provide the necessary contact for providing the torque to seat the electrode into the ventricular septum or other tissue for electrically connecting to the cardiac conduction system. In an embodiment, a core (e.g., a cone-shaped component, not shown) can be disposed within the helical space of the second electrode 220. In another embodiment, there can be no core within the helical space of the second electrode 220. The core can be of either conductive or nonconductive.

Referring back to FIG. 1, the shocking coil 180 (or 280) is provided along the lead body 110 spaced away from the second electrode 120 or the distal end. In an embodiment, the shocking coil 180 is provided at a distance between at or about 10 mm to at or about 50 mm from the second electrode 120 or the distal end, or longer. As such, when a portion of the lead is implanted in the IVS through the right ventricle, the lead body 110 can have a curvature, e.g., due to the flexibility of the lead body and length of the slack or portion between the shocking coil 180 and to near the proximal end of the second electrode 120, such that the curvature is configured to reduce force exerted on the CSP pacing electrodes implanted in the IVS. As such, when the second electrode 120 or the electrode 220 is attached to the portion of the ventricular septum, for example, at least partly inside the right ventricular septum, the shocking coil 180 is positioned in the right ventricle (RV) at a different position, e.g., inferior to, than the second electrode 120 or the electrode 220, for example, in the right ventricle and/or along the wall of the right ventricular septum, as will be further discussed below.

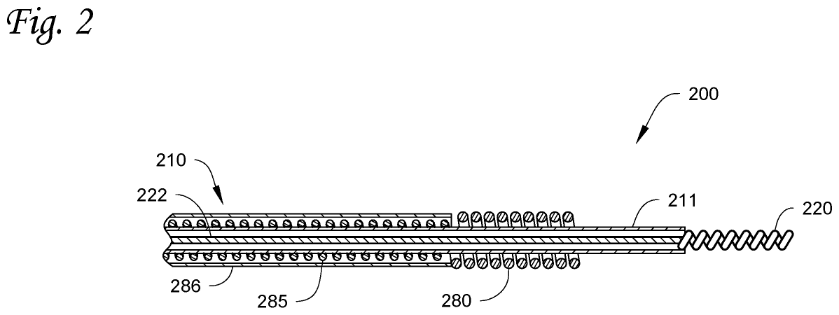

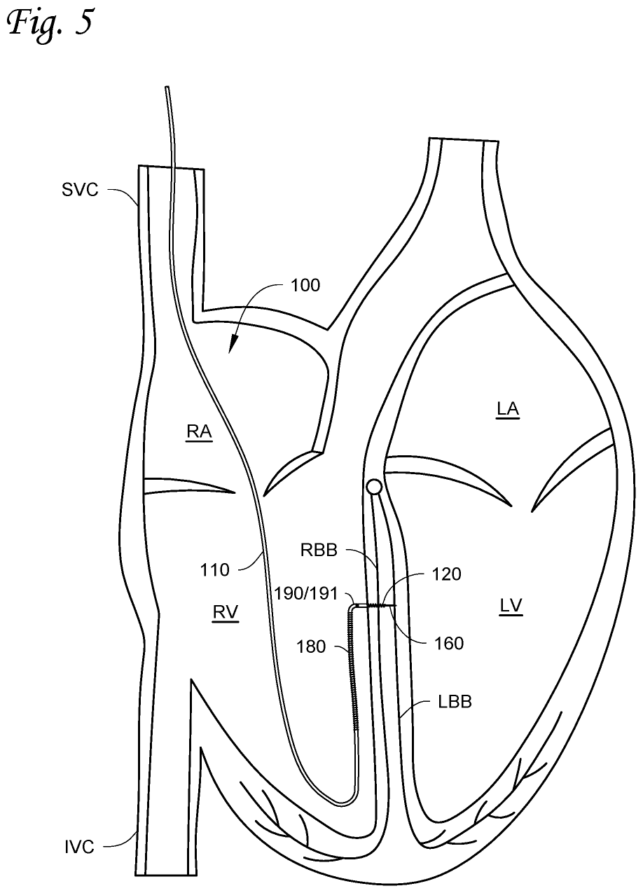

In an embodiment, the lead 100 includes a stress release portion 190, for example, as shown in FIGS. 3 and 5, provided between the shocking coil 180 and the second electrode 120, e.g., a transitioning portion or slack when implanted between the shocking coil 180 and the second electrode 120. The stress release portion 190 is configured to relieve stress, e.g., pressure or force, exerted on the CSP pacing electrodes, e.g., the first electrode and the second electrode, that are implanted in the ventricular septum, for example, due to the heavier weight of the RV coil and/or gravity effects of the lead body 110 and/or force caused by the movement of the heart during heart beats. As such, the stress release portion 190 can provide and/or improve chronic stability and prevent or reduce the likelihood of the dislodgement of the first electrode and/or the second electrode to prevent or mitigate perforation of the tissue.

In an embodiment, the stress release portion 190 is made of a softer or more flexible material than the remainder of the lead body, for example, a flexible polymer or plastic or biocompatible metal, between the shocking coil 180 and the second electrode 120 that allows the bending between the second electrode 120 and the portion of the lead body 110 having the shocking coil 180. In another embodiment, the stress release portion 190 includes a hinge or hinge-like portion (for example, with a very soft/flexible and flex-fatigue resistant segment) between the shocking coil 180 and the second electrode 120, in which a pivot is provided to allow the flexible bending of the lead body 110 at that location without transferring unacceptable forces to the CSP electrodes, e.g., unacceptable forces that result in lead electrode dislodgement or injury to the myocardium at the implantation location resulting in an unstable or elevated pacing threshold.

In an embodiment, as seen in FIG. 3, the stress release portion 190 can be a preformed section 191 between the shocking coil 180 and the second electrode 120 that is provided to provide a specific positioning of the shocking coil 180, e.g., positioned along and “supported” by the ventricular septum when the CSP pacing electrodes of lead 100 are implanted in the ventricular septum to electrically capture the cardiac conduction system. A terminal pin 195 or any suitable control mechanism is provided at the proximal end of the lead 100 which can be rotated to drive the inner coil to extend distally or retract the first electrode. The preformed section 191 is provided at an angle θ between at or about 65 degrees and at or about 105 degrees, and preferably at an angle of at or about 90 degrees. As such, when a portion of the lead 100 is implanted into the ventricular septum by inserting the first lead electrode 160 and attaching the second lead electrode 120 into the ventricular septum, the stress release portion 190 relieves forces provided on the connection of the CSP electrodes at the distal portion of the lead to the ventricular septum, e.g., less downward force from the weight of the lead and shocking coil 180, e.g., due to gravity. In an embodiment, fluoroscope, ultrasound or other imaging modalities can be performed to measure the length of the ventricular septum and to determine and/or select the desired length of the stress release portion for the placement of the shocking coil 180.

It is appreciated that while the stress release portion 190 has been discussed herein with respect to specific examples, the examples are provided as exemplary examples and not intended to limit the scope of the disclosure. Rather, other structures for the stress release portion 190 can be used that allow certain portions of the lead body 110 and the shocking coil 180 to be provided at different positions than the second electrode 120.

In an embodiment, an outer diameter of the first electrode 160 (the linear electrode) can be smaller than an inner diameter of the second electrode 120 (the helix electrode), such that the first electrode 160 and the second electrode 120 are co-axial, and/or that the first electrode 160 is disposed in the helical space of the second electrode 120. The first electrode 160 can have a length of at or about four millimeters. The second electrode 120 can have a length of at or about four millimeters. As such, as seen in FIGS. 1A-1E, the first electrode 160 is provided in an internal space of the second electrode 120 and retracted in the lead body 110 such that after the second electrode 120 is attached to the ventricular septum, the first electrode 160 can be extended for further insertion into the ventricular septum so that the electrodes of the lead 100 can reach the respective conduction pathway(s), e.g., His-bundle, RBB, LBB, etc., of the cardiac conduction system.

The implantation and operation of the lead for cardiac conduction system pacing with defibrillation capabilities is discussed below.

As disclosed in U.S. application Ser. No. 17/804,705, during the implantation procedure, the catheter can be inserted to reach a specific portion of the septum or other tissue. When the desired location is determined, the lead 100 can be inserted through an orifice extending from a proximal end of the catheter to the distal end of the catheter. The distal end of the lead 100 can be placed to the desired location using the catheter or stylet. The catheter and/or the stylet can then be removed. In an embodiment, the lead 100 can be delivered using e.g., at or about or larger than a 6 French inner diameter sheath or catheter, a guide wire, and/or a stylet.

It will be appreciated that positioning the catheter against the septum can include one or more of the steps of pulling the first deflection wire to deflect the distal end of the catheter, pulling the second deflection wire to further deflect the distal end of the catheter, and positioning a tip of the distal end of the catheter to be perpendicular to an endocardial surface of the septum.

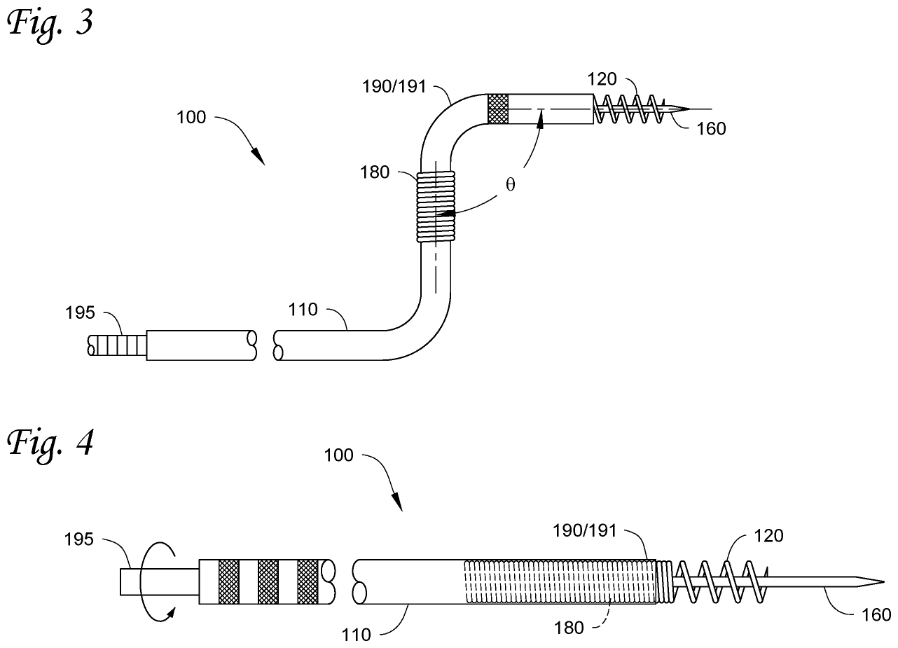

As illustrated in FIG. 4, the lead 100 can be operated to place the electrodes in the ventricular septum at the desired location to electrically capture the cardiac conduction system. For example, in an embodiment, the lead 100 can be rotated such that the second electrode 120, e.g., helix electrode, penetrates the heart tissue (e.g., the ventricular septum), while the first electrode 160 is in a retracted state. For example, in an embodiment, when the second electrode 120 touches the tissue, the lead 100 can be rotated (e.g., in a clockwise or counterclockwise direction) to pose/dispose the second electrode 120 into the heart tissue.

After the second electrode 120 is inserted into the heart tissue, the first electrode 160, e.g., a linear electrode, of the lead 100 can be inserted into the ventricular septum by extending the first electrode 160 from the retracted state to the extended state from the lead body 110. In an embodiment, a terminal pin 195 (e.g., an IS-1 pin, DF4, or other type of control) or any suitable control mechanism at the proximal end of the lead 100 can be rotated to extend and deploy the first electrode 160 into the ventricular septum (e.g., so that the electrodes of the lead 100 can reach the respective conduction pathway(s), e.g., His-bundle, RBB, LBB, etc., of the cardiac conduction system).

As illustrated in FIG. 5, in an embodiment, after the first electrode 160 is deep-seated into the ventricular septum and the second electrode 120 is inserted into the ventricular septum, e.g., to be electrically captured in the respective conduction pathways, i.e., RBB with second electrode 120 and LBB with first electrode 160, the stress release portion 190 is configured such that the shocking coil 180 is positioned against or along the RV septum and/or in the blood pool in the right ventricle. In an embodiment, the stress release portion 190 is flexible such that the weight of the lead 100 and shocking coil 180 bends the lead at the stress release portion 190, e.g., due to gravity. In another embodiment, the stress release portion 190 has a preformed section 191, such that after the first electrode 160 and the second electrode 120 are attached to the ventricular system, the stress release portion 190 may position the shocking coil 180 at certain angles, e.g., 90 degrees, so that the shocking coil 180 is provided against or along the RV septum or at certain positions in the right ventricle. As such, since at least a portion of the lead 100 that includes the shocking coil 180 is placed at a different position, e.g., along the RV septum wall, from the first electrode 160 and the second electrode 120, the lead 100 is configured to provide stability and/or prevent or mitigate dislodgement and possible tearing or perforation of the tissue due to gravity and/or due to movements of the heart, e.g., heart beats.

It is appreciated that at least in view of the structure of the lead 100, as described herein, the lead 100 has a number of advantageous benefits.

For example, in an embodiment, the lead 100 is structured for deep seating the first electrode, which can overcome the disadvantages of conventional lead designs for ease of implantation and securement and placement into the ventricular septum. As such, the embodiments disclosed herein can help to easily place the lead deep into the interventricular septum (i.e., the ventricular septum) to target/locate the conduction pathway(s) such as LBB, can reduce (or produce less) heart tissue trauma and may result in a lower and stable pacing threshold, and/or can provide secured lead attachment with chronic lead stability. That is, the embodiments disclosed herein can provide better attachment (e.g., when the second electrode 120 is screwed into the ventricular septum) and better electrical performance than a conventional lead, and can facilitate ease of the lead 100 being deep seated (being inserted deep) into and can facilitate attachment of the lead onto the ventricular septum (e.g., the second electrode being at or around the RBB inside the septum and the first electrode being deep seated at or around the LBB inside the septum).

In an embodiment, in which the shocking coil 180 is in the right ventricle, e.g., a RV coil, the bursts of energy for shocking is delivered to the heart to stop a fast or rapid beating of the heart, e.g., ventricular fibrillation.

This CSP lead with defibrillation capability may also provide an alternative therapy for chronic heart failure patients, especially patients indicated for the CRT-D therapy.

Moreover, in an embodiment, the lead 100 is configured to be adjustable to provide different combinations of pacing vectors, e.g., the components can be used for different functions than discussed above to provide the pacing, sensing, shocking, etc. For example, in an embodiment, the shocking coil 160 can be used as the anode and the second electrode 120 can be used as the cathode. As such, the lead 100 is configured such that IPG can deliver the bursts of energy to second electrode 120 for pacing, etc.

Aspects: It is appreciated that any one of the aspects can be combined with other aspect(s).

Aspect 1: A lead for cardiac conduction system pacing that has defibrillation capability, the lead comprising a lead body; a distal end including a first electrode configured to be inserted into a portion of a ventricular septum; a shocking coil mounted on the lead body and spaced away from the second electrode; and a proximal end.

Aspect 2: The lead according to Aspect 1, wherein first electrode is a linear electrode and the distal end comprises a spacer connected to the linear electrode, and wherein the spacer is adjustable to distally extend or proximally retract the linear electrode.

Aspect 3: The lead according to Aspect 2, wherein the proximal end is configured to be rotated to adjust the spacer to distally extend or proximally retract the linear electrode.

Aspect 4: The lead according to any of Aspects 1-3, further comprising a second electrode coupled to the lead body and a fixation element configured to fix the lead to the portion of the ventricular septum

Aspect 5: The lead according to any of Aspects 1-4, wherein the second electrode is a helix electrode and includes the fixation element that is configured to be fixed into the portion of the ventricular septum by a rotation of the lead into the portion of the ventricular septum.

Aspect 6: The lead according to any of Aspects 1-5, wherein the lead further comprises a stress release portion between the shocking coil and the distal end such that the shocking coil is spaced away from the distal end.

Aspect 7: The lead according to Aspect 6, wherein the stress release portion is flexible such that the stress release portion is configured so that the shocking coil is provided at a different position than the second electrode when attached to the portion of the ventricular septum.

Aspect 8: The lead according to Aspect 6, wherein the stress release portion is a preformed section such that the shocking coil is provided at an angle from the second electrode.

Aspect 9: The lead according to Aspect 9, wherein the angle is between at or about 65 degrees and at or about 105 degrees.

Aspect 10: The lead according to Aspect 0, wherein the angle is at 90 degrees.

Aspect 11: The lead according to any of Aspects 1-10, wherein the first electrode is a cathode and the second electrode is an anode.

Aspect 12: The lead according to any of Aspects 1-10, wherein the second electrode is a cathode and the shocking coil is an anode.

Aspect 13: The lead according to any of Aspects 1-12, wherein the shocking coil is spaced away from the distal end at a distance between at or about 10 mm and at or about 50 mm.

Aspect 14: The lead according to any of Aspects 1-13, wherein the shocking coil has a diameter smaller than a diameter of an outer surface of the lead.

Aspect 15: The lead according to any of Aspects 1-13, wherein the shocking coil has a diameter a same size as or larger than a diameter of an outer surface of the lead.

Aspect 16: The lead according to any one of Aspects 14-15, wherein the diameter of the shocking coil is between at or about 5 French and at or about 9 French.

Aspect 17: The lead according to any of Aspects 1-16, wherein the shocking coil has a surface area between at or about 350 mm² and at or about 650 mm².

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

What is claimed is:

1. A lead for a cardiac conduction system pacing that has defibrillation capability, the lead comprising:
a lead body;
a distal end including a first electrode configured to be inserted into a portion of a ventricular septum;
a shocking coil mounted on the lead body and spaced away from the second electrode; and
a proximal end.

2. The lead according to claim 1, wherein first electrode is a linear electrode and the distal end comprises a spacer connected to the linear electrode, and wherein the spacer is adjustable to distally extend or proximally retract the linear electrode.

3. The lead according to claim 2, wherein the proximal end is configured to be rotated to adjust the spacer to distally extend or proximally retract the linear electrode.

4. The lead according to claim 1, further comprising a second electrode coupled to the lead body and a fixation element configured to fix the lead to the portion of the ventricular septum.

5. The lead according to claim 4, wherein the second electrode is a helix electrode and includes the fixation element that is configured to be fixed into the portion of the ventricular septum by a rotation of the lead into the portion of the ventricular septum.

6. The lead according to claim 1, wherein the lead further comprises a stress release portion between the shocking coil and the distal end such that the shocking coil is spaced away from the distal end.

7. The lead according to claim 6, wherein the stress release portion is flexible such that the stress release portion is configured so that the shocking coil is provided at a different position than the distal end when attached to the portion of the ventricular septum.

8. The lead according to claim 6, wherein the stress release portion is a preformed section such that the shocking coil is provided at an angle from the distal end.

9. The lead according to claim 8, wherein the angle is between at or about 65 degrees and at or about 105 degrees.

10. The lead according to claim 9, wherein the angle is at or about 90 degrees.

11. The lead according to claim 4, wherein the first electrode is a cathode and the second electrode is an anode.

12. The lead according to claim 4, wherein the second electrode is a cathode and the shocking coil is an anode.

13. The lead according to claim 1, wherein the shocking coil is spaced away from the distal end at a distance between at or about 10 mm and at or about 50 mm.

14. The lead according to claim 1, wherein the shocking coil has a diameter smaller than a diameter of an outer surface of the lead.

15. The lead according to claim 1, wherein the shocking coil has a diameter that is a same size as or larger than a diameter of an outer surface of the lead.

16. The lead according to claim 13, wherein the diameter of the shocking coil is between at or about 5 French and at or about 9 French.

17. The lead according to claim 1, wherein the shocking coil has a surface area between at or about 350 mm² and at or about 650 mm².
